(19) United States
(12) Patent Application Publication
(10) Pub. No.: US 2026/0123971 A1
(43) Pub. Date: May 7, 2026

(54) DEVICES AND METHODS FOR SPINAL IMPLANT REMOVAL

(71) Applicant: KIC VENTURES, LLC, BURLINGTON, MA (US)

(72) Inventors: VITO LORE, BURLINGTON, MA (US); JOSHUA FINKEL-LOPEZ, BURLINGTON, MA (US); KINGSLEY R. CHIN, BURLINGTON, MA (US)

(73) Assignee: KIC VENTURES, LLC, BURLINGTON, MA (US)

(21) Appl. No.: 19/382,537

(22) Filed: Nov. 7, 2025

Related U.S. Application Data
(60) Provisional application No. 63/717,614, filed on Nov. 7, 2024.

(51) Int. Cl. A61B 17/92; A61B 17/56; A61F 2/46

(52) U.S. Cl. CPC A61B 17/92; A61F 2/4611; A61B 2017/564; A61B 2017/925; A61F 2002/4619; A61F 2002/4681

(57) ABSTRACT

A device for spinal implant removal includes a driver tool and a capture tool. The driver tool includes an elongated cylindrical body with a through-opening and two distal end protrusions. The capture tool includes an elongated shaft configured to fit and extend through the cylindrical body through-opening of the driver tool and has a distal end opening with inner threads. The spinal implant includes a cylindrical body and first and second sets of wings that are configured to engage first and second spinous processes of two vertebrae, respectively. The cylindrical body houses a threaded post that actuates the deployment of the first and second sets of wings. The two distal end protrusions of the driver tool are configured to engage a circular collar formed around the threaded post on a top surface of the cylindrical body. The driver tool with the engaged circular collar are rotated counter clockwise to screw the threaded post out of the cylindrical body and thereby to release the first and second sets of wings. The elongated shaft of the capture tool is configured to be rotated counter clockwise to capture outer threads of the threaded post with the inner threads of the distal end.

### CROSS REFERENCE TO RELATED CO-PENDING APPLICATIONS

This application claims the benefit of U.S. provisional application Ser. No. 63/717,614 filed Nov. 7, 2024 and entitled “DEVICES AND METHODS FOR SPINAL IMPLANT REMOVAL”, the contents of which are expressly incorporated herein by reference.

### FIELD OF THE INVENTION

The present invention relates to devices and methods for spinal implant removal, and in particular, to devices, instruments, and methods that limit the size of the incision, and provide safe and accurate targeting and removal of a spinal implant.

### BACKGROUND OF THE INVENTION

The human spine is comprised of individual vertebrae 30 that are connected to each other to form a spinal column 29, shown in FIG. 1A. Referring to FIG. 1A, FIG. 1B, FIG. 1C, each vertebra 30 has a cylindrical bony body (vertebral body) 32, three winglike projections (two transverse processes 33, 35 and one spinous process 34), left and right facet joints 46, lamina 47, left and right pedicles 48 and a bony arch (neural arch) 36. Each vertebral body 32 has top and bottom endplates 32a, 32b, respectively. The bodies of the vertebrae 32 are stacked one on top of the other and form the strong but flexible spinal column 29. The neural arches 36 are positioned so that the space they enclose forms a tube, i.e., the spinal canal 37. The spinal canal 37 houses and protects the spinal cord and other neural elements. The spinal column 29 is flexible enough to allow the body to twist and bend, but sturdy enough to support and protect the spinal cord and the other neural elements. The vertebrae 30 are separated and cushioned by thin pads of tough, resilient fiber known as inter-vertebral discs 40. There is a small opening (foramen) 42 between each vertebra 30, through which nerves 44 pass and go to different body parts.

Under normal circumstances the structures that make up the spine function are configured to protect the neural structures, allow us to stand erect, bear axial loads, and are flexible for bending and rotation. Disorders of the spine occur when one or more of these spine structures are abnormal. In these pathologic circumstances, surgery may be tried to restore the spine to the normal state and to relieve the patient of pain. Some spinal pathologies, such as spinal stenosis, require spinal decompression surgery, which involves the insertion of an interspinous implant 140 between two vertebrae 30a, 30b, as shown in FIG. 2A-FIG. 2C, in order to distract and decompress the vertebrae 30a, 30b and to restrict extension of the spine. Implant 140 usually remains in the patient's body. However, in some cases, implant removal surgery is performed to remove the decompressing or stabilizing components that were previously implanted in the spine. Spinal implant removal surgery is recommended when the implant causes pain, irritation, allergic reaction, or infection, or if it has moved from its original position, or was misplaced and needs to be repositioned, or there is an implant failure. Implant removal is also recommended when there are long term implant side-effects, such as facet osteoarthritis, decreased mobility, disc degeneration, metallic toxicity, infections or other allergic reaction.

When a patient's spine implant needs to be altered or removed, it is recommended to use a tool set from the same company that produced the implant that was used in the implant insertion surgery. However, obtaining the tool set used in the initial surgery may not always be feasible. Furthermore, the tool set used for insertion of the spinal implant is not always suitable for removing the spinal implant. Accordingly, there is a need for new devices, instruments, and methods that can engage and remove the Superion™ implant, while limiting the size of the incision, and providing safe and accurate targeting and removal of the implant.

### SUMMARY OF THE INVENTION

The present invention relates to devices and methods for spinal implant removal, and in particular, to devices, instruments, and methods that can engage and remove a spinal implant, while limiting the size of the incision, and providing safe and accurate targeting and removal of a previously inserted implant.

In general, in one aspect, the invention features a device for spinal implant removal including a driver tool and a capture tool. The driver tool includes an elongated cylindrical body with a through-opening and two distal end protrusions. The capture tool includes an elongated shaft configured to fit and extend through the cylindrical body through-opening of the driver tool and has a distal end opening with inner threads. The spinal implant includes a cylindrical body and first and second sets of wings that are configured to engage first and second spinous processes of two vertebrae, respectively. The cylindrical body houses a threaded post that actuates the deployment of the first and second sets of wings. The two distal end protrusions of the driver tool are configured to engage a circular collar formed around the threaded post on a top surface of the cylindrical body. The driver tool with the engaged circular collar are rotated counter clockwise to screw the threaded post out of the cylindrical body and thereby to release the first and second sets of wings. The elongated shaft of the capture tool is configured to be rotated counter clockwise to capture outer threads of the threaded post with the inner threads of the distal end.

Implementations of this aspect of the invention may include one or more of the following features. The device further includes a cannula comprising an elongated body with a through-opening and two distal end protrusions, and the two distal end protrusions of the cannula are configured to engage two opposite recesses formed on an outer surface of the cylindrical body of the implant and the driver tool is configured to be inserted into the cannula through-opening in order to reach the implant. The device further includes a cannula comprising an elongated body with a through-opening and two distal end protrusions, and the two distal end protrusions of the cannula are configured to wedge between an outer surface of the cylindrical body and the spinous processes. The cannula further includes a distal end stop. The device further includes a slap hammer comprising an elongated shaft surrounded by a sliding weight, and a distal end of the elongated shaft is configured to engage a proximal end of the capture tool. The distal end of the slap hammer comprises a channel that houses a spring plunger.

In general, in another aspect, the invention features a method for spinal implant removal including providing a driver tool comprising an elongated cylindrical body with a through-opening and two distal end protrusions. Next, providing a capture tool comprising an elongated shaft configured to fit and extend through the cylindrical body through-opening of the driver tool and comprising a distal end opening with inner threads. Next, inserting the driver tool into a vertebral location where the spinal implant is located. The spinal implant comprises a cylindrical body and first and second sets of wings that are configured to engage first and second spinous processes of two vertebrae, respectively, and wherein the cylindrical body houses a threaded post that actuates the deployment of the first and second sets of wings. Next, engaging the two distal end protrusions of the driver tool to a circular collar formed around the threaded post on a top surface of the cylindrical body. Next, rotating the driver tool with the engaged circular collar counter clockwise to screw the threaded post out of the cylindrical body and thereby to release the first and second sets of wings. Next, inserting the elongated shaft of the capture tool into the vertebral location where the spinal implant is located through the through-opening of the cylindrical body of the driver tool, and then rotating the elongated shaft of the capture tool counter clockwise to capture outer threads of the threaded post with the inner threads of the distal end. Next, pulling a proximal end of the elongated shaft of the capture tool to remove the implant, the capture tool and the driver tool.

The details of one or more embodiments of the invention are set forth in the accompanying drawings and description below. Other features, objects, and advantages of the invention will be apparent from the following description of the preferred embodiments, the drawings, and the claims.

### BRIEF DESCRIPTION OF THE DRAWINGS

Referring to the figures, wherein like numerals represent like parts throughout the several views:

FIG. 1A is a side view of the human spinal column;

FIG. 1B is an enlarged view of area A of FIG. 1A;

FIG. 1C is an axial cross-sectional view of a lumbar vertebra;

FIG. 2A is a side view of the spinal column with the inserted spinal implant;

FIG. 2B is a perspective view of the spinal column with the inserted spinal implant;

FIG. 2C is an enlarged front view of the spinal column area with the inserted spinal implant;

FIG. 3A depicts a perspective view of the Superion™ spinal implant with the wings in the closed position;

FIG. 3B depicts a side view of the implant of FIG. 3A;

FIG. 3C depicts a perspective view of the Superion™ spinal implant with the wings in the open position;

FIG. 3D depicts a front view of the implant of FIG. 3C;

FIG. 3E depicts a back view of the implant of FIG. 3C;

FIG. 3F depicts a cross-section of the implant of FIG. 3B;

FIG. 4A depicts a perspective top view of the implant driver tool according to this invention;

FIG. 4B depicts a perspective bottom view of the implant driver tool of FIG. 4A;

FIG. 4C depicts the enlarged distal end of the implant driver tool of FIG. 4A;

FIG. 4D depicts a front view of the implant driver tool of FIG. 4A;

FIG. 4E depicts a side view of the implant driver tool of FIG. 4A;

FIG. 4F depicts a cross-sectional view of the implant driver tool of FIG. 4A along the BB′ plane;

FIG. 4G depicts a top view of the implant driver tool of FIG. 4A;

FIG. 5A depicts a top perspective view of the set screw (or threaded post) capture tool according to this invention;

FIG. 5B depicts a bottom perspective view of the set screw capture tool of FIG. 5A;

FIG. 5C depicts a front view of the set screw capture tool of FIG. 5A;

FIG. 5D depicts a top view of the set screw capture tool of FIG. 5A;

FIG. 5E depicts a bottom view of the set screw capture tool of FIG. 5A;

FIG. 5F depicts cross-sectional view of the set screw capture tool of FIG. 5A along the AA′ plane;

FIG. 6A depicts a top perspective view of the slap hammer tool according to this invention;

FIG. 6B depicts a bottom perspective view of the slap hammer tool of FIG. 6A;

FIG. 6C depicts a front view of the slap hammer tool of FIG. 6A;

FIG. 6D depicts a side view of the slap hammer tool of FIG. 6A;

FIG. 6E depicts a cross-sectional view of the slap hammer tool of FIG. 6A along the EE′ plane;

FIG. 7A depicts a top perspective view of the cannula tool according to this invention;

FIG. 7B depicts a front view of the cannula tool of FIG. 7A;

FIG. 7C depicts an enlarged view of the distal end of the cannula tool of FIG. 7A;

FIG. 7D depicts a bottom view of the cannula tool of FIG. 7A;

FIG. 7E depicts a side view of the cannula tool of FIG. 7A;

FIG. 7F depicts a cross sectional view of the cannula tool of FIG. 7E along the DD′ plane;

FIG. 8A depicts the positioning of the patient during spinal implant removal surgery;

FIG. 8B depicts the incision on the patient's lumbar area for the spinal implant removal surgery;

FIG. 8C depicts the step of bringing the implant driver tool of FIG. 4A near the implant;

FIG. 8D depicts the step of bringing the distal end of the implant driver tool of FIG. 4A close to the set screw drive geometry of the implant;

FIG. 8E depicts the step of engaging the distal end of the implant driver tool of FIG. 4A to the set screw drive geometry of the implant;

FIG. 8F depicts the step of inserting the set screw capture tool of FIG. 5A through the implant driver tool of FIG. 4A;

FIG. 8G depicts the step of bringing the distal end of the set screw capture tool of FIG. 5A near the set screw of the implant;

FIG. 8H depicts the step of engaging the distal end of the set screw capture tool of FIG. 5A to the set screw of the implant;

FIG. 8I depicts the step of releasing the wings of the implant;

FIG. 8J depicts the step of removing the implant with the closed the wings from the interspinous location;

FIG. 9A depicts the step of bringing the slap hammer close to the proximal end of the set screw capture tool of FIG. 5A;

FIG. 9B depicts the step of bringing the distal end of the slap hammer close to the proximal end of the set screw capture tool of FIG. 5A;

FIG. 9C depicts the step of engaging the distal end of the slap hammer close to the proximal end of the set screw capture tool of FIG. 5A;

FIG. 9D depicts the engaged slap hammer to the set screw capture tool of FIG. 5A;

FIG. 9E depicts the engaged and pulled slap hammer in order to remove the implant from the interspinous location;

FIG. 10A depicts the step of bringing the distal end of the cannula tool near the implant location;

FIG. 10B depicts the step of engaging the distal end of the cannula tool to the cylindrical main body of the implant;

FIG. 11A is a side perspective view of another embodiment of the cannula tool;

FIG. 11B is a top perspective view of the cannula tool of FIG. 11A with the inserted driver tool;

FIG. 11C is a perspective view of the distal end of the cannula tool of FIG. 11A with the inserted driver tool;

FIG. 11D is a bottom perspective view of another embodiment of the driver tool;

FIG. 11E depicts a top perspective view of another embodiment of the capture tool inserted into the driver tool of FIG. 11D and the cannula of FIG. 11A;

FIG. 11F depicts a bottom perspective view of the distal end of the implant removal device of FIG. 11E;

FIG. 11G depicts a perspective view of the distal end of the driver tool of FIG. 11D and the inserted capture tool of FIG. 11E;

FIG. 11H depicts a cross-sectional view of the distal end of the implant removal device of FIG. 11C;

FIG. 11I depicts a perspective view of another embodiment of the slap hammer engaged to the handle of the capture tool of FIG. 11E;

FIG. 11J depicts a perspective view of the pulled capture tool of FIG. 11E;

FIG. 11K depicts a side view of the distal end of the slap hammer near the handle of the capture tool of FIG. 11E;

FIG. 11L depicts a side view of the engaged distal end of the slap hammer to the handle of the capture tool of FIG. 11E;

FIG. 12 depicts a flow diagram of the process of removing a spine implant according to this invention.

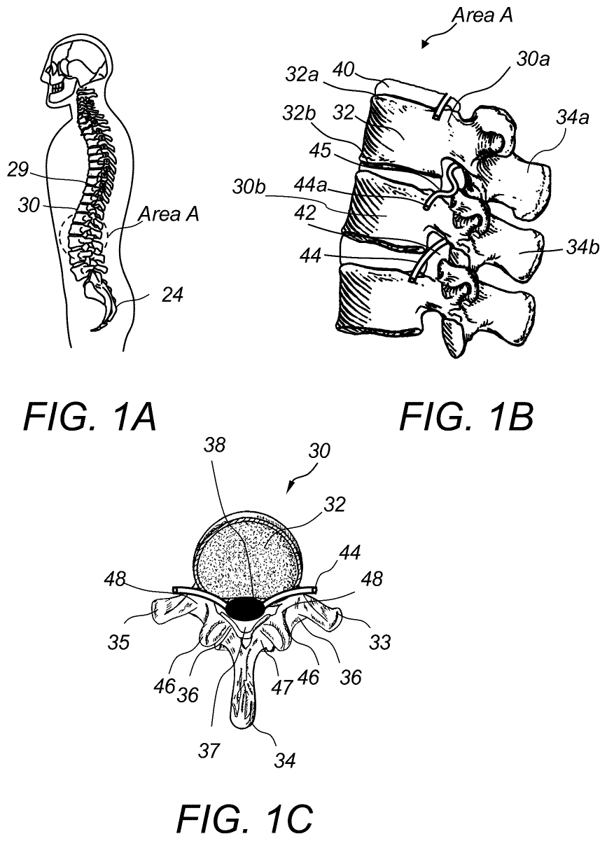

### DETAILED DESCRIPTION OF THE INVENTION

The present invention relates to devices and methods for implant removal, and in particular, to devices, instruments, and methods that can engage and remove an implant, while limiting the size of the incision, and providing safe and accurate targeting and removal of a previously inserted implant.

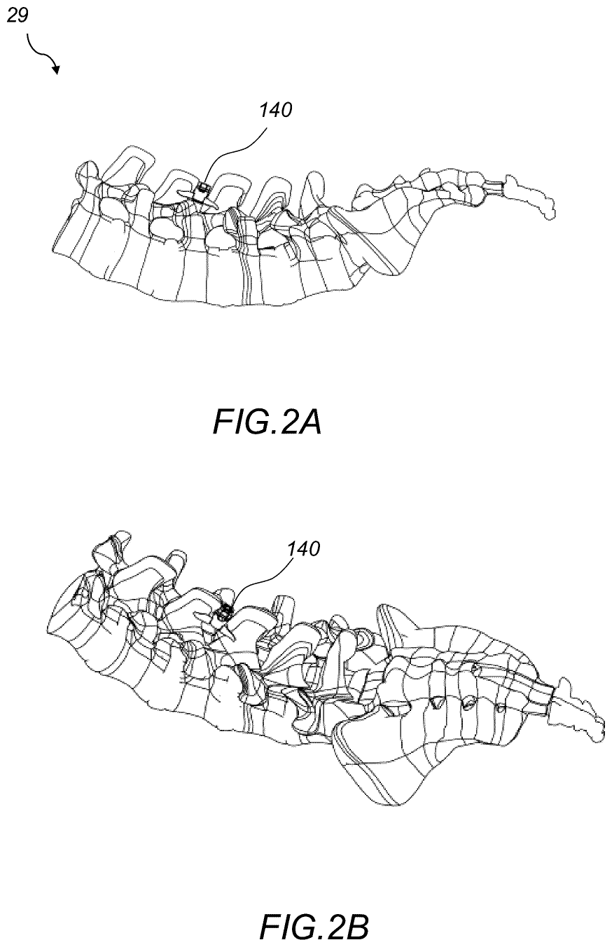

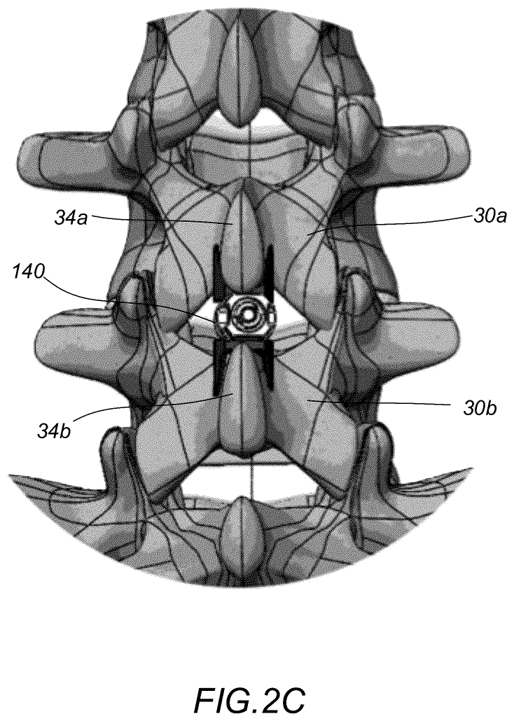

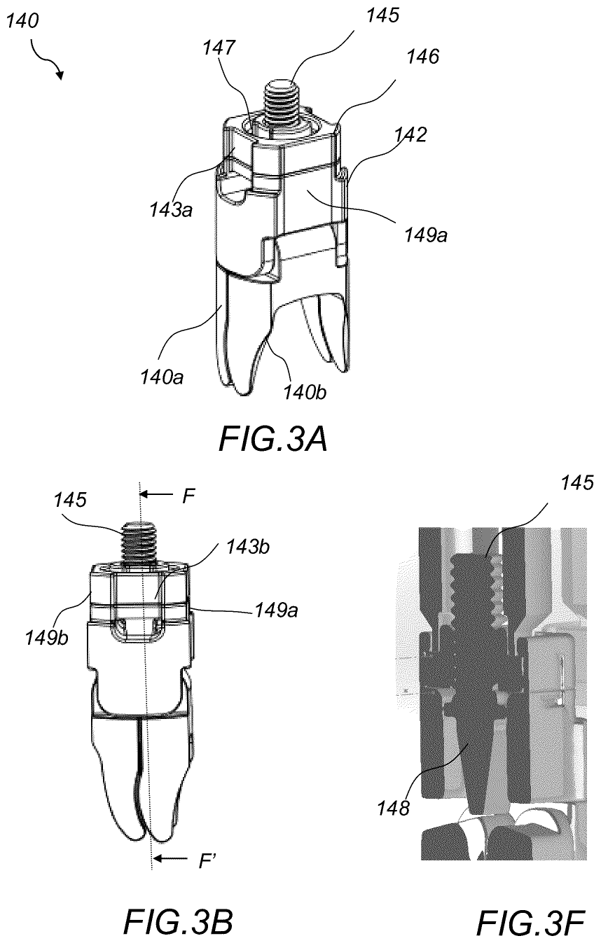

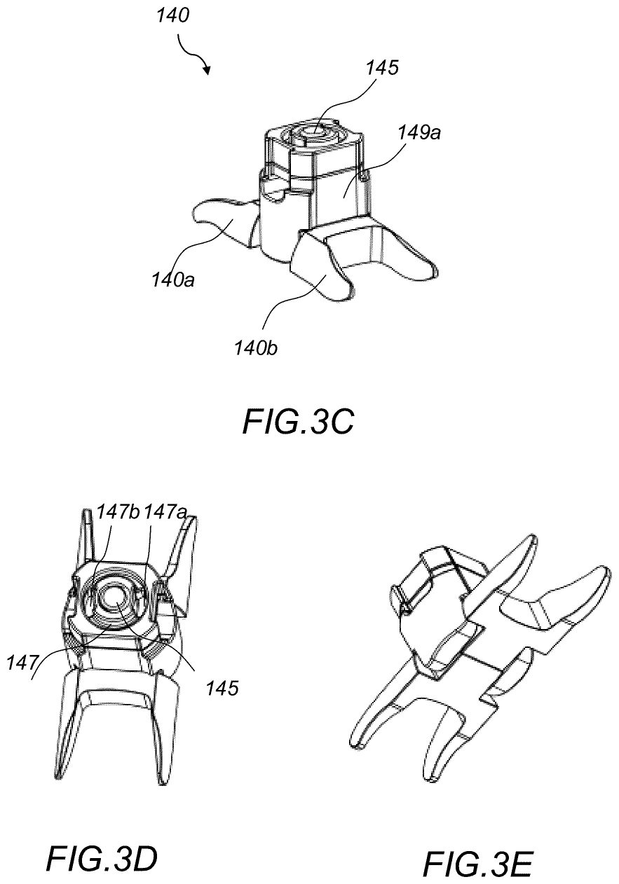

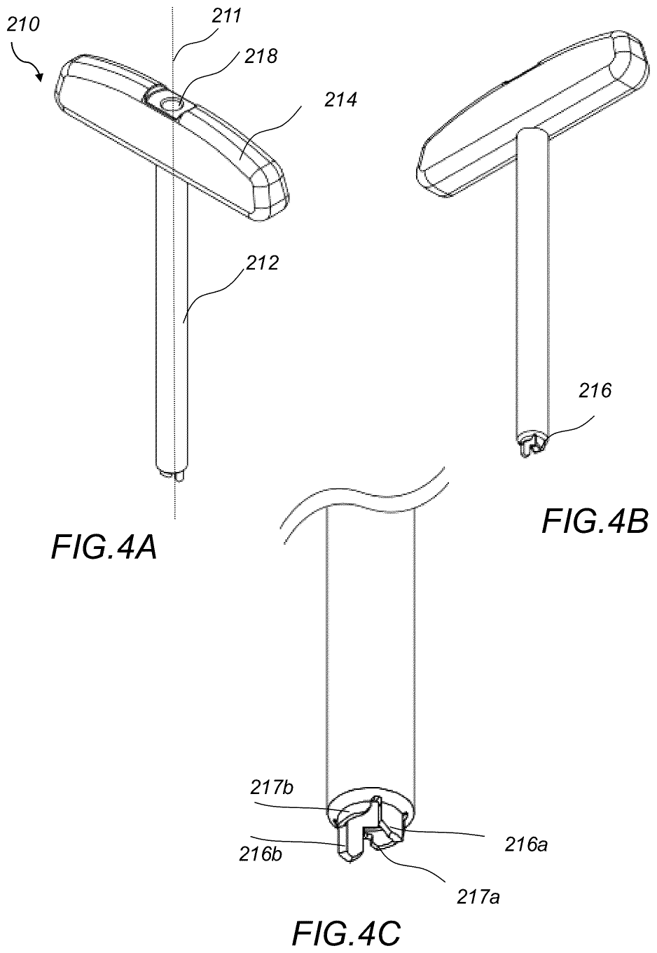

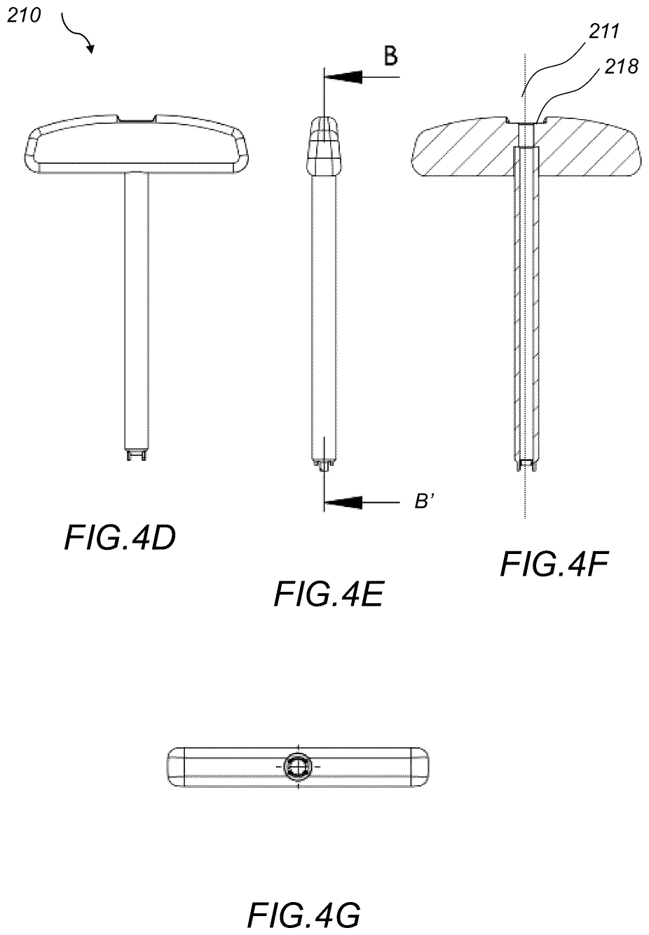

As was mentioned above, some spinal pathologies, such as spinal stenosis, require spinal decompression surgery, which involves the insertion of an interspinous implant 140 between two vertebrae 30a, 30b, as shown in FIG. 2A-FIG. 2C. Several interspinous implant systems exist for decompressing the spine, reducing pressure on the spinal nerves and improve stability in the lower back. In one example, implant 140 is the Superion™ indirect decompression system that is manufactured by Boston Scientific Corporation. The Superion™ implant is described in U.S. Pat. No. 9,532,812, entitled “Interspinous Spacer”, the contents of which are incorporated herein by reference. Referring to FIG. 3A-FIG. 3F, implant 140 includes a cylindrical body 142 and two deployable set of wings 140a, 140b. The cylindrical body 142 houses a threaded post 145 that translates with an affixed wedge 148 for opening and closing of the wings 140a, 140b. Threaded post 145 is surrounded by a collar 147 the has two recesses 147a, 147b, that are sized to receive two protruding fingers 216a, 216b of the driver tool 210, as shown in FIG. 4C and will be described below. The cylindrical body 142 also includes two opposite recesses 143a, 143b, on two opposite outer surfaces, and two opposite flat surfaces 149a, 149b above the wings 140a, 140b. Implant 140 is inserted percutaneously between the spinous processes 34a, 34b of the two vertebrae 30a, 30b with the wings 140a, 140b, in the closed position. Subsequently, the collar 147 is rotated clockwise with the driver tool and the threaded post 145 is screwed into the cylindrical body 142 and the wings 140a, 140b, are opened to engage the spinous processes 34a, 34b, respectively, as shown in FIG. 2C and FIG. 3C, in order to distract and decompress the vertebrae 30a, 30b and to restrict extension of the spine. The opposite procedure needs to be followed in order to remove the implant 140, during implant removal surgery. For releasing the wings 140a, 140b, the collar 147 is rotated counter-clockwise with the driver tool and the threaded post 145 is screwed out of the cylindrical body 142, as shown in FIG. 3A.

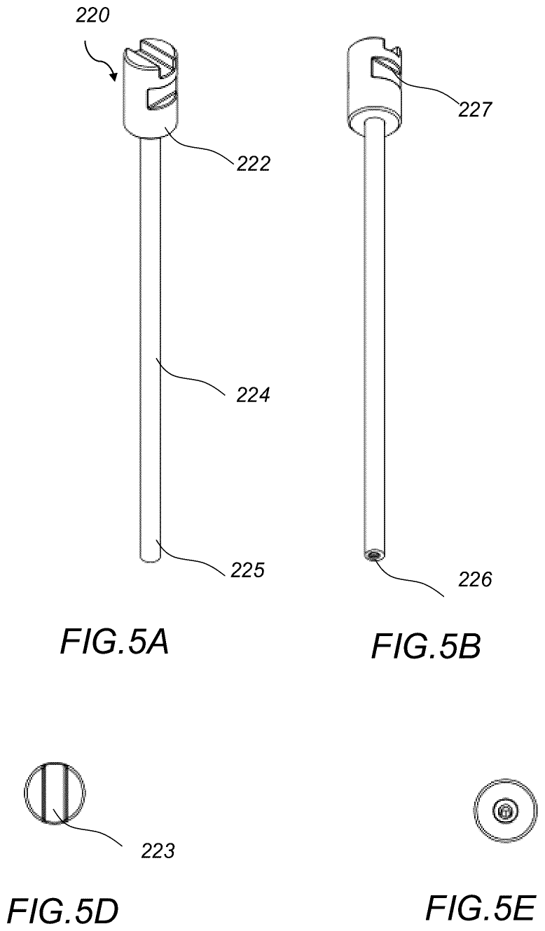

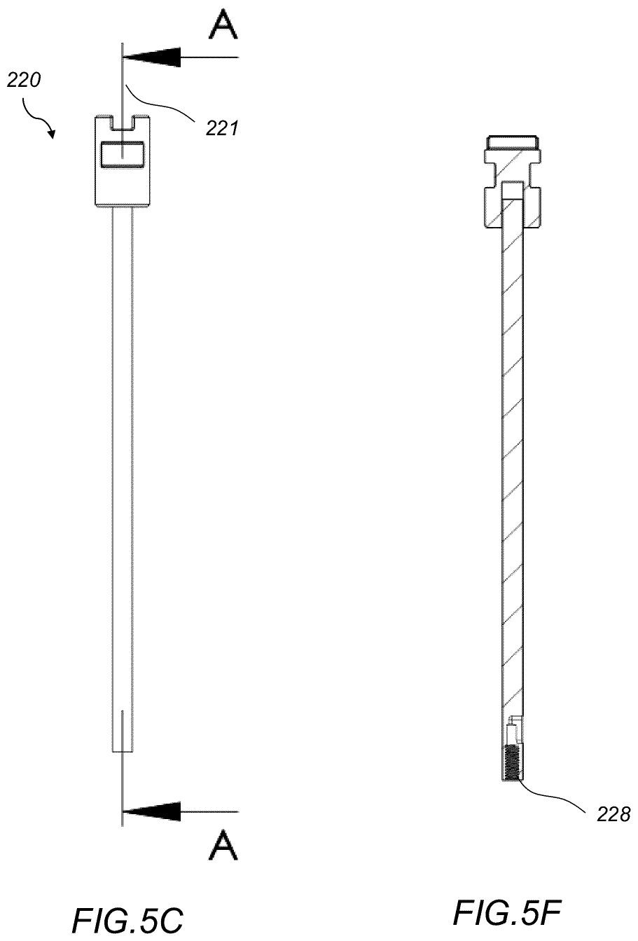

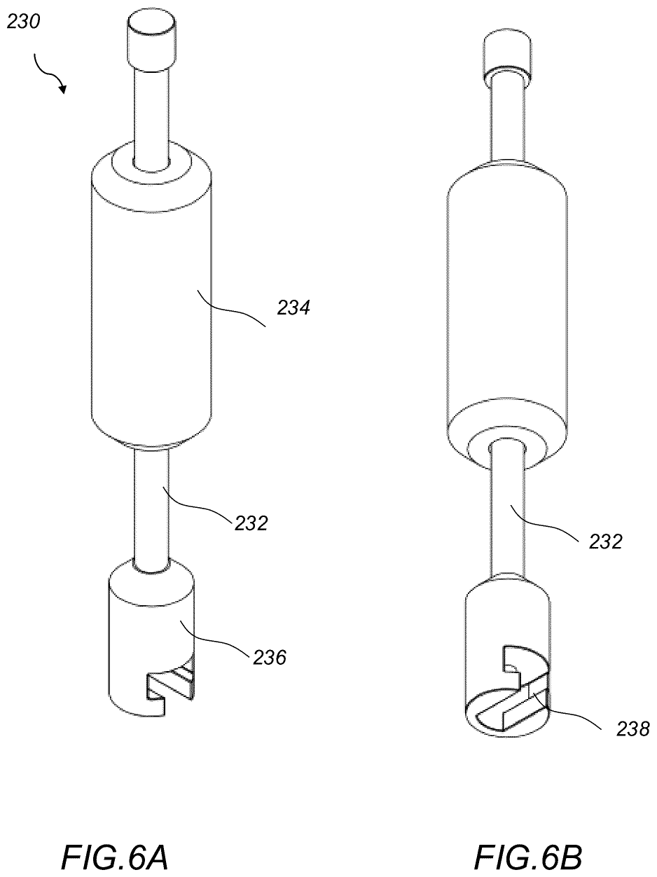

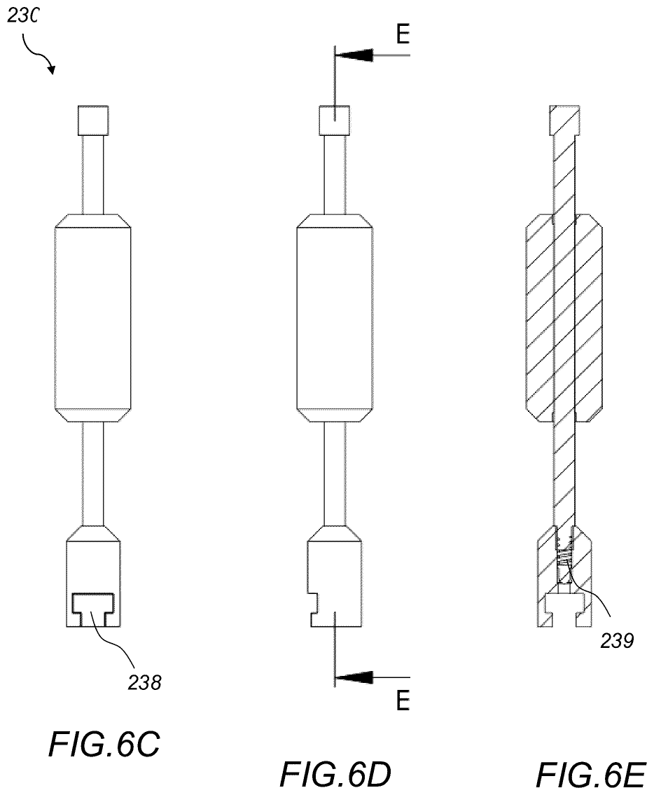

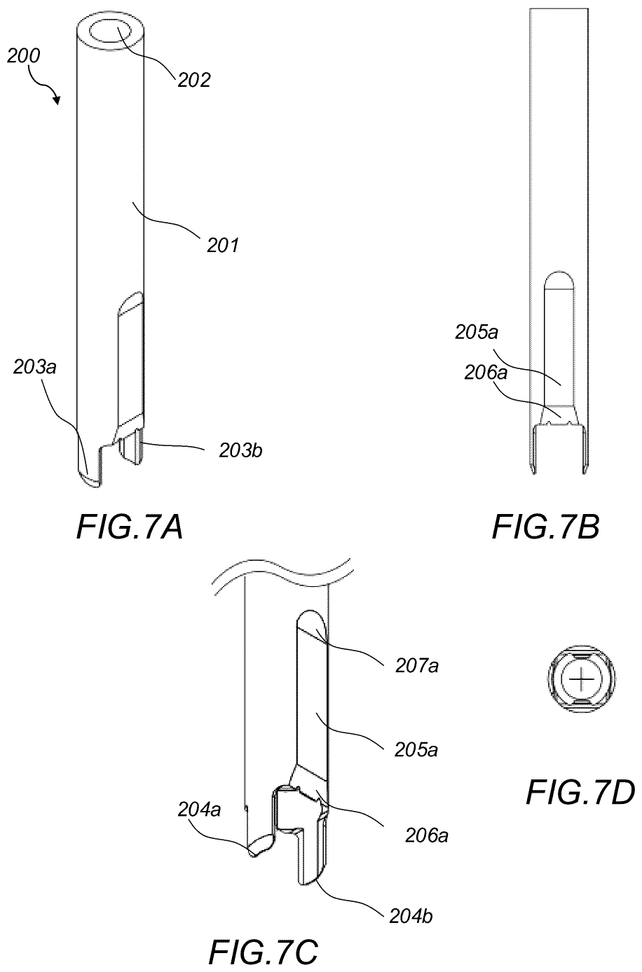

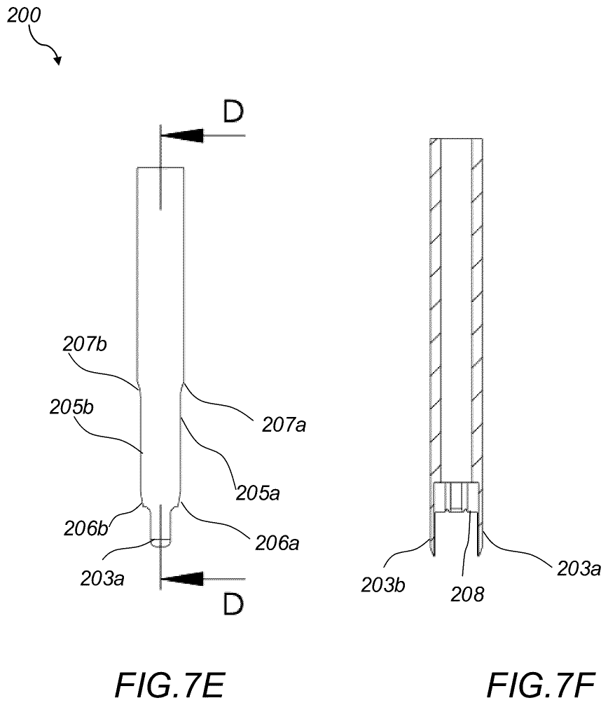

The tools for removing the implant 140 include a driver tool, a capture tool, and optionally a slap hammer tool and a cannula tool. Referring to FIG. 4A-FIG. 4G, driver tool 210 includes a cylindrical body 212 having a handle 214 at the proximal end and two protruding fingers 216a, 216b, at the distal end 216. Distal end 216 also includes semi-circular extensions 217a, 217b. Handle 214 includes a through opening 218 extending through the cylindrical body 212 along axis 211. Referring to FIG. 5A-FIG. 5G, the capture tool 220 includes an elongated cylindrical body 224 having a cylindrical head 222 at the proximal end and a threaded inner surface 226 at the distal end 225. Head 222 has a slotted top opening 223 and a side opening 227. The elongated cylindrical body 224 of the capture tool 220 is sized to fit through the through opening 218 of the driver tool 210 and exit through the distal end of 216. The cylindrical head 222 of the capture tool 220 is dimensioned to remain above the handle 214 of the driver tool 210. Referring to FIG. 6A-FIG. 6E, the slap hammer tool 230 includes an elongated cylindrical rod 232 with a distal end component 236. A cylindrical-shaped weight 234 surrounds and slides along the rod 232. The inertia of the sliding weight 234 is transferred to the rod 232 and is used to pull the engaged capture tool head 222 and the implant 140. The distal end component 236 has a cutout opening 238 that is configured to receive and engage the capture tool head 222. The cutout opening 238 of the distal end of the slap hammer 230 has a channel onto which a spring plunger 239 is located. Referring to FIG. 7A-FIG. 7F, cannula 200 has an elongated cylindrical body 201 with a through-opening 202. Cannula 200 includes two finger-like protrusions 203a, 203b, extending from opposite sides of the distal end of the cannula. Protrusions 203a, 203b are rectangular shaped and terminate in rounded conical tips 204a, 204b, respectively. The distal end also includes two opposite flat surfaces 206a, 206b that terminate in angled ends 205a, 205b, respectively. Angled ends 205a, 205b are designed to wedge between the spinous processes 34a, 34b and the flat surfaces 149a, 149b of the implant 140 above the wings 140a, 140b, respectively. The proximal ends 207a, 207b of flat surfaces 206a, 206b are also angled. Cannula 210 also includes a stop 208 at the distal end that interfaces with the top surface of the implant body 142 to prevent the cannula from reaching the spinal cord.

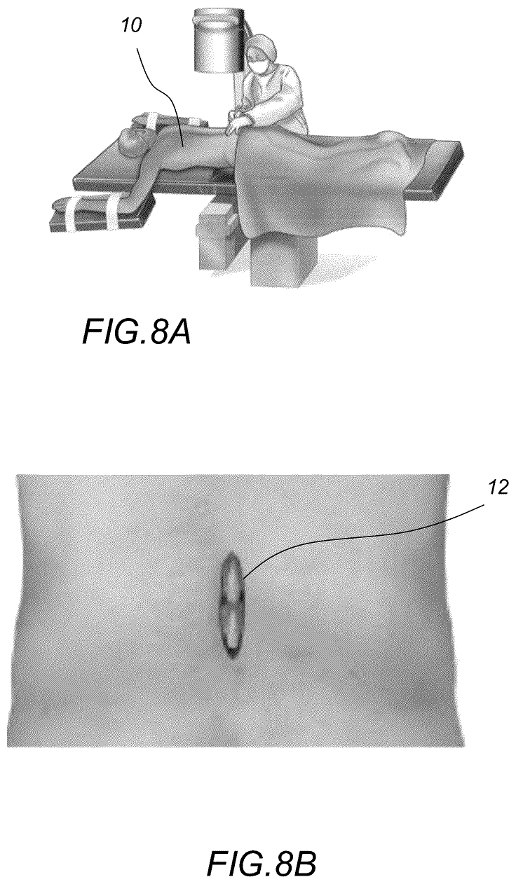

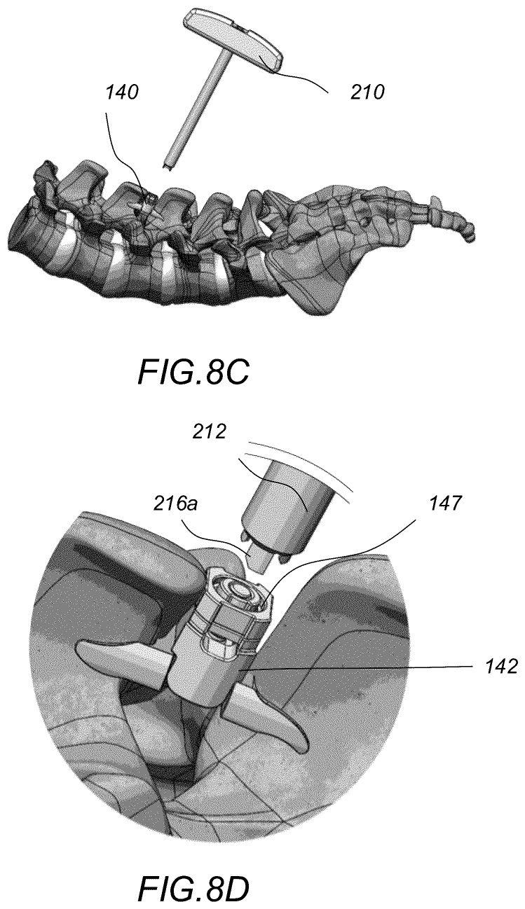

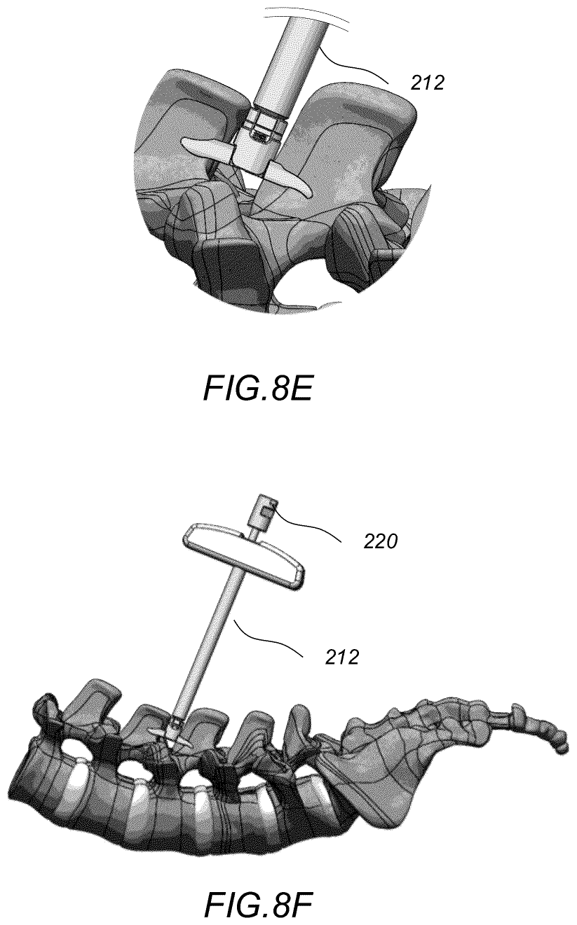

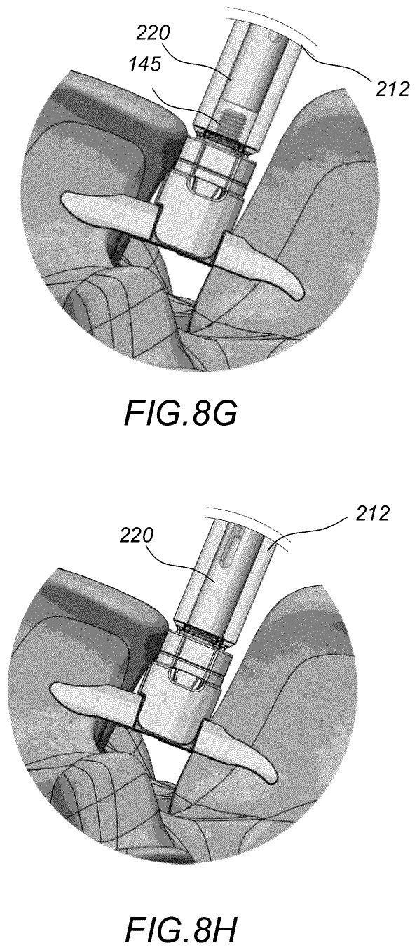

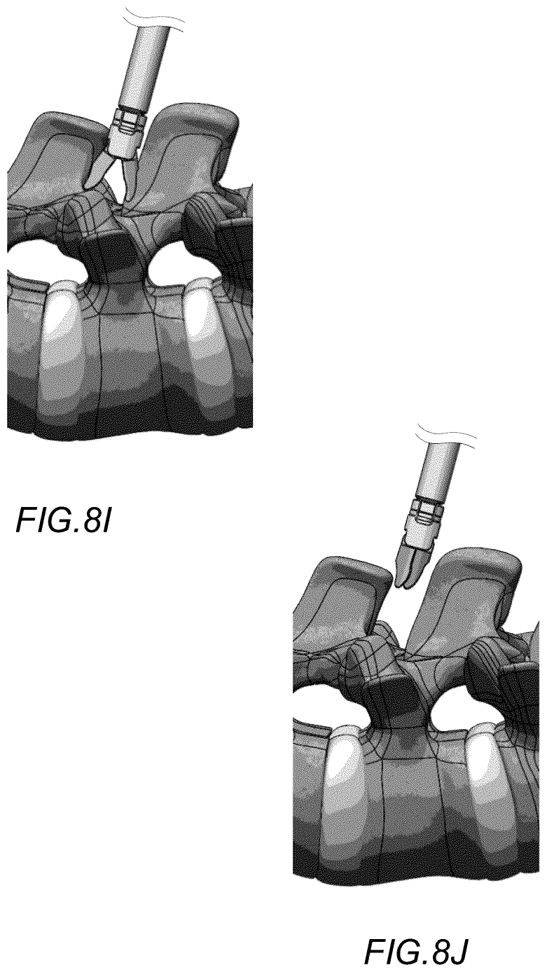

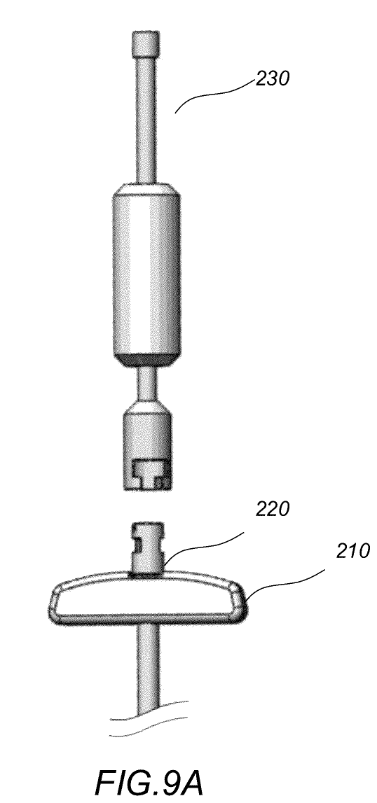

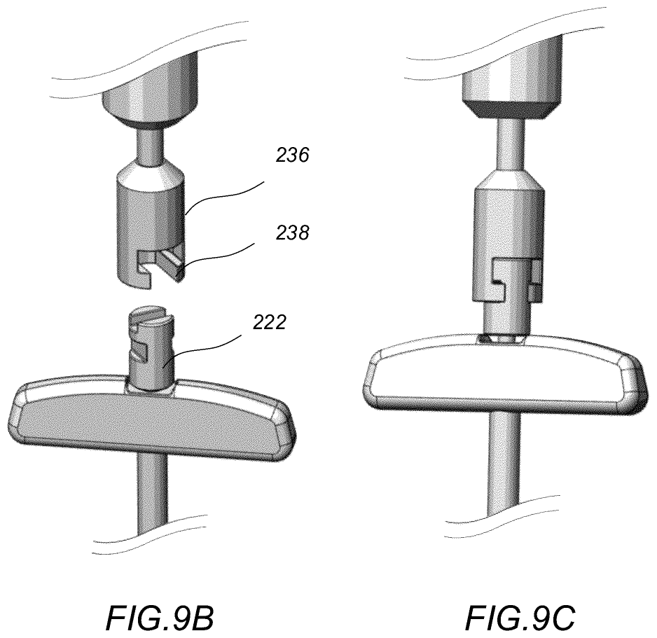

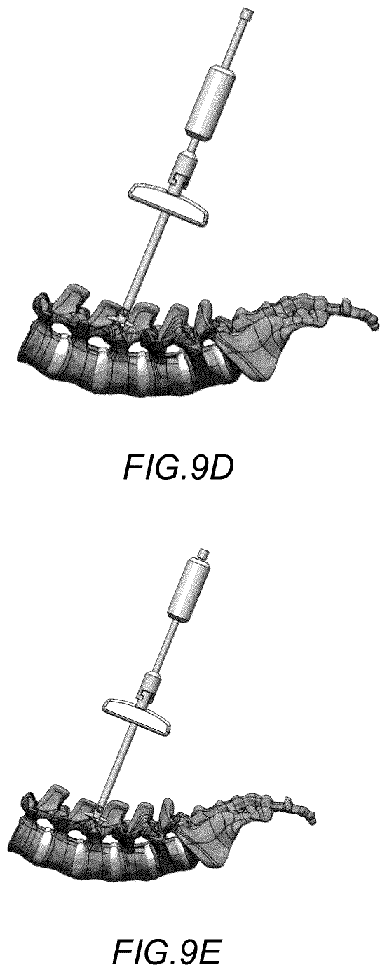

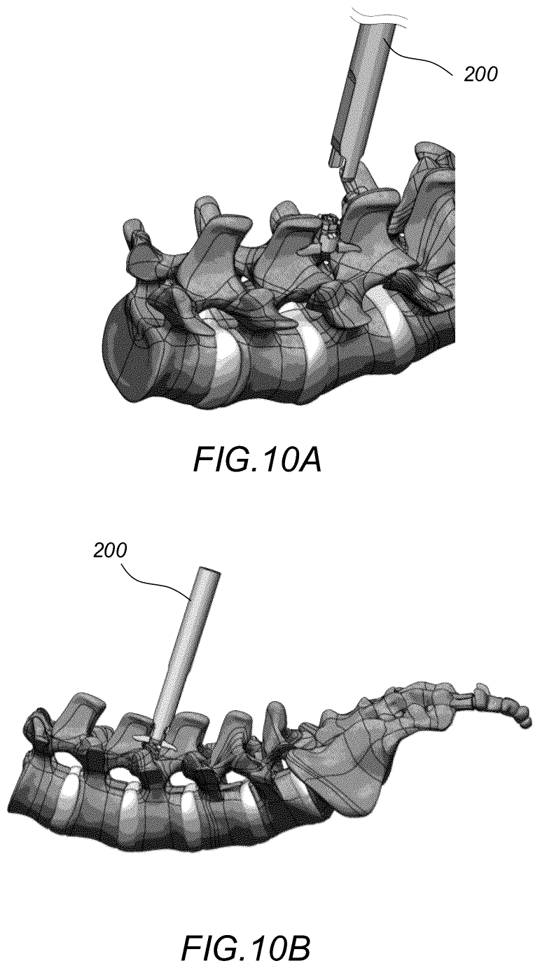

Referring to FIG. 8A-FIG. 8J, during implant removal surgery the patient 10 is positioned in prone position to provide access to the surgical site and an incision 12 is made. Next, the driver tool 210 is inserted near the implant 140, as shown in FIG. 8C, and then downward force is applied onto the driver so that the two protruding fingers 216a, 216b are inserted into the two recesses 147a, 147b of the circular collar 147 on the top surface of the of the implant cylindrical body 142. Next, the circular collar 147 is rotated counter clockwise with the driver tool 210 until it stops, thereby unscrewing the threaded post 145 out of the cylindrical body 142 and releasing the wings 140a, 140b from the spinous processes 34a, 34b, respectively. Next, the capture tool 220 is inserted through the through-opening 218 of the driver tool 210 and is forced down onto the implant cylindrical body 142, and then the capture tool is rotated counter-clockwise to engage outer threads of the threaded post 145 of the implant 140. Next, the driver tool 210 with the inserted capture tool 220 and the engaged implant 140 are pulled out proximally until the implant wings 140a, 140b, collapse and close, and then the entire assembly is removed from the surgical site though the incision opening 12. The procedure is guided via direct visualization and fluoroscopy. A combination of anterior-posterior (AP), lateral, and oblique fluoroscopic views are utilized to localize the vertebral anatomy and to guide the positioning of instruments.

In some cases, a cannula tool 200 is used to create a working portal for the removal of soft tissue prior and for inserting the driver and capture tools. Cannula 200 is mounted on top of the cylindrical body 142 of the implant so that the distal protrusions 203a, 203b contour to the cylindrical body 142 and slide past the end of it, on the outer surface of the implant cylindrical body 142, thus providing alignment with the implant device. Cannula stop 208 at the distal end interfaces with the top surface of the implant body 142 to prevent the cannula 200 from reaching the spinal cord. If additional force is needed to remove the driver tool 210 with the inserted capture tool 220 and the engaged implant 140, a slap hammer 230 may be used. The capture tool head 222 is inserted into the cutout opening 238 of the distal end of the slap hammer 230 to engage the proximal end of the capture tool 220, and then the cylindrical-shaped weight 234 of the slap hammer 230 is slid along the rod 232 and is used to pull the entire assembly out of the surgical site. The cutout opening 238 of the distal end of the slap hammer 230 has a channel onto which a spring plunger 239 is located. The spring plunger on the slap hammer ensures axial alignment with the capture tool 220.

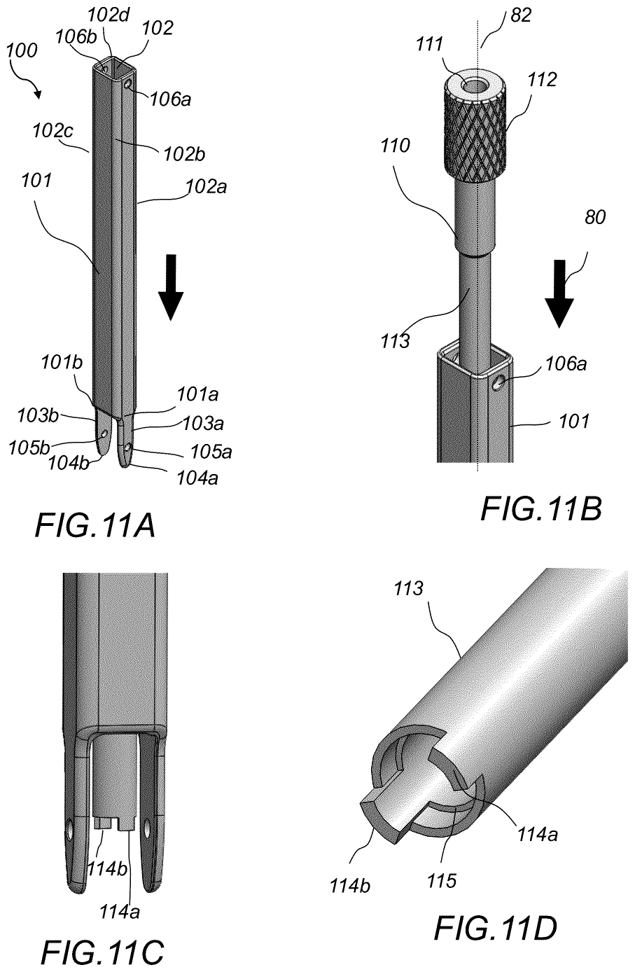

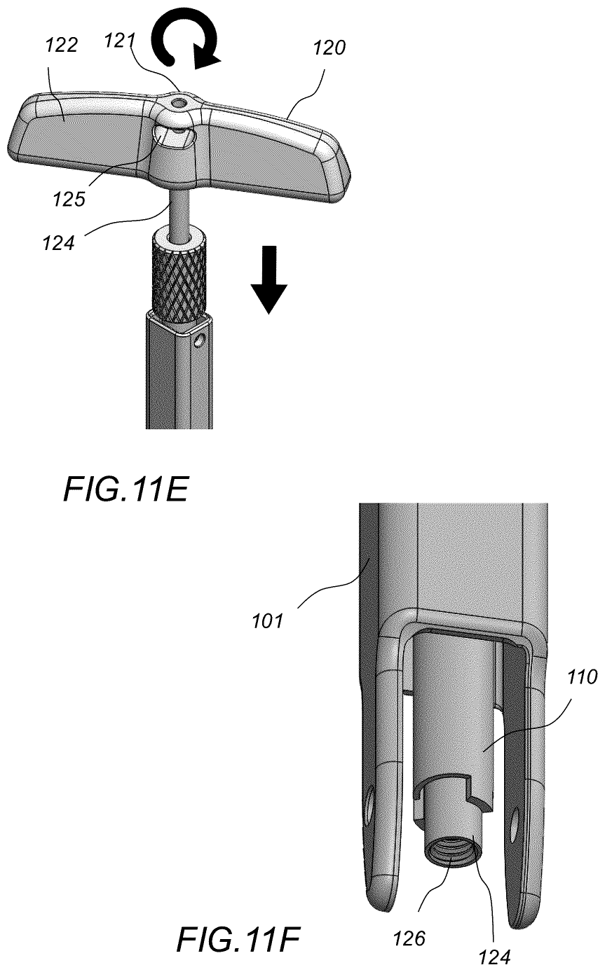

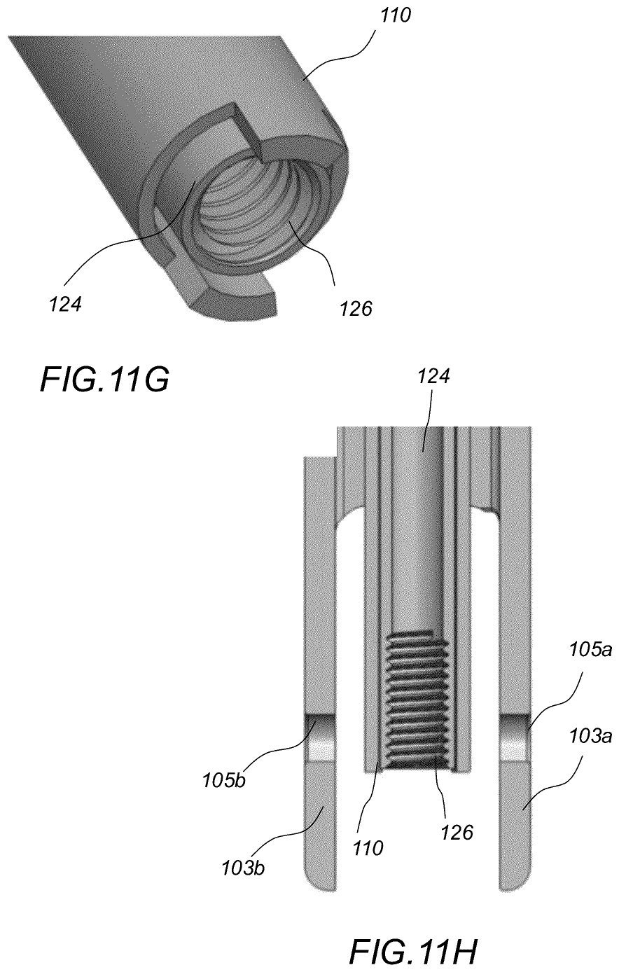

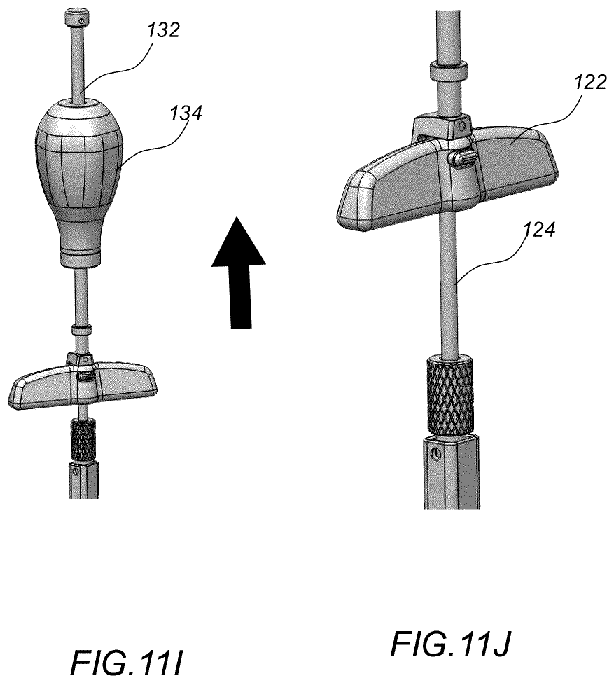

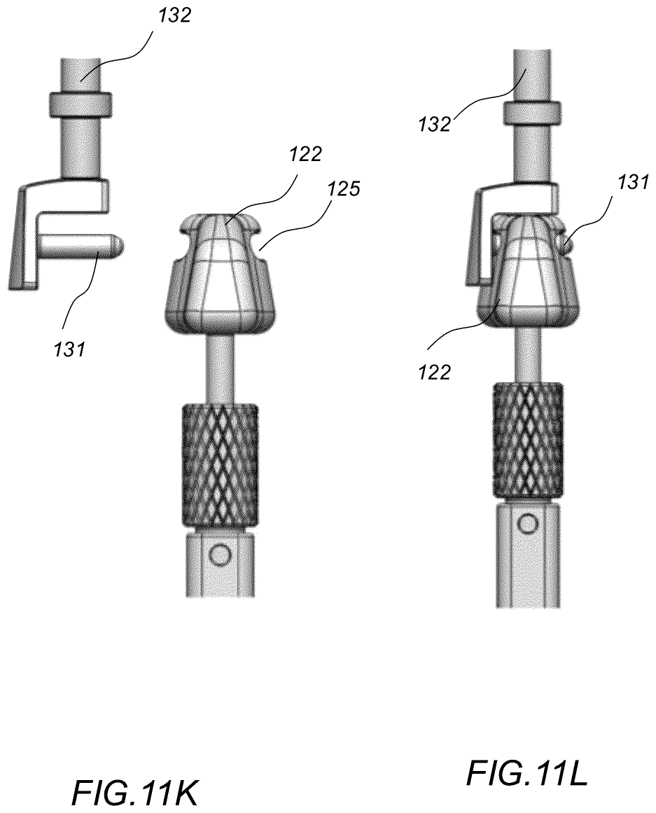

Referring to FIG. 11A-FIG. 11L, in another embodiment, a square cannula tool 100 is inserted under fluoroscopic visualization into a vertebral location where the implant is located and is directed toward the implant. Cannula 100 has an elongated body 101 with a rectangular cross-section through-opening 102 and rounded edges 102a-102d. Cannula 100 includes two finger-like protrusions 103a, 103b, extending from opposite sides 101a, 101b, of the distal end of the cannula. Protrusions 103a, 103b are rectangular shaped and terminate in rounded conical tips 104a, 104b, respectively. Each finger-like protrusion 103a, 103b includes an opening 105a, 105b, respectively. The opposite cannula sides 101a, 101b of the cannula also include openings 106a, 106b, respectively, at the proximal end of the cannula. Proximal end openings 106a, 106b are aligned with the distal end openings 105a, 105b, respectively. As was mentioned above, in one example, implant 140 includes a cylindrical body 142 and two deployable wings 140a, 140b. The cylindrical body 142 houses a threaded post 145 with an affixed wedge 148 driver mechanism that activates the opening and releasing/closing of the wings 140a, 140b. The cylindrical body 142 includes two opposite recesses 143a, 143b, on the outer surface. The two finger-like protrusions 103a, 103b of cannula 101 are sized and shaped to engage the two opposite recesses 143a, 143b of the cylindrical body 142, thereby docking cannula 101 onto the cylindrical body 142.

Next, a driver tool 110 is inserted into the square cannula 101 and advanced along direction 80 through the opening 102 of the cannula tool 100 and exits through the distal end of the cannula 101 and is positioned and docked onto the cylindrical body 142, as shown in FIG. 8C. Tool 110 is rotated around axis 82 until it stops. Tool 110 includes an elongated rod body 113 with a knurled proximal end 112, and a through-opening 111 extending though body 113 along axis 82. The distal end of rod 113 includes two opposite protrusions 114a, 114b extending downwards along axis 82, and an inset 115. Protrusions 114a, 114b are shaped and dimensioned to engage recesses 147a, 147b formed on the collar 147 around the threaded post 145. Next, the circular collar 147 is rotated counter clockwise with the driver tool 110 until it stops, thereby unscrewing the threaded post 145 out of the cylindrical body 142 and releasing the wings 140a, 140b from the spinous processes 34a, 34b, respectively. Next, a capture tool 120 is inserted into the through-opening 111 of tool 110 and is rotated counter clockwise onto the threaded post 145 to capture it. Capture tool 120 includes an elongated rod body 124 and a T-handle 122 located at the proximal end of elongated rod 124. Elongated rod body 124 includes through-opening 121 and has inner screw threads 126 at the distal end 124 of the through-opening 121. The inner screw threads 126 are used to engage outer threads of the threaded post 145. T-handle 122 also includes a horizontal through opening 125 that is shaped and dimensioned to receive a protruding component 131 of a slap hammer 130, as shown in FIG. 11K-FIG. 11L. In this embodiment, the T-handle 122 is integrated with (or fixedly attached to) the proximal end of the elongated rod body 124. In other embodiments, handle 122 is modular and is removably attached to the proximal end of the elongated rod body 124.

Next, a slap (or slide) hammer 130 is used to engage the T-handle of the capture tool 120 by inserting a protrusion 131 into the opening 125 of the T-handle 122 until it stops, as shown in FIG. 11K-FIG. 11L. As was mentioned above, the capture tool 120 is engaged to the implant 140 via the threaded post 145. Slap hammer 130 includes an elongated cylindrical rod 132 with a distal component 136. An orb-shaped weight 134 surrounds the rod 132 and is used to slide along the rod 132 and to pull the rod with the engaged implant 140. The distal component 136 has the protrusion 131 that is configured to be inserted into the opening 125 of the T-handle 122 of the capture tool 120.

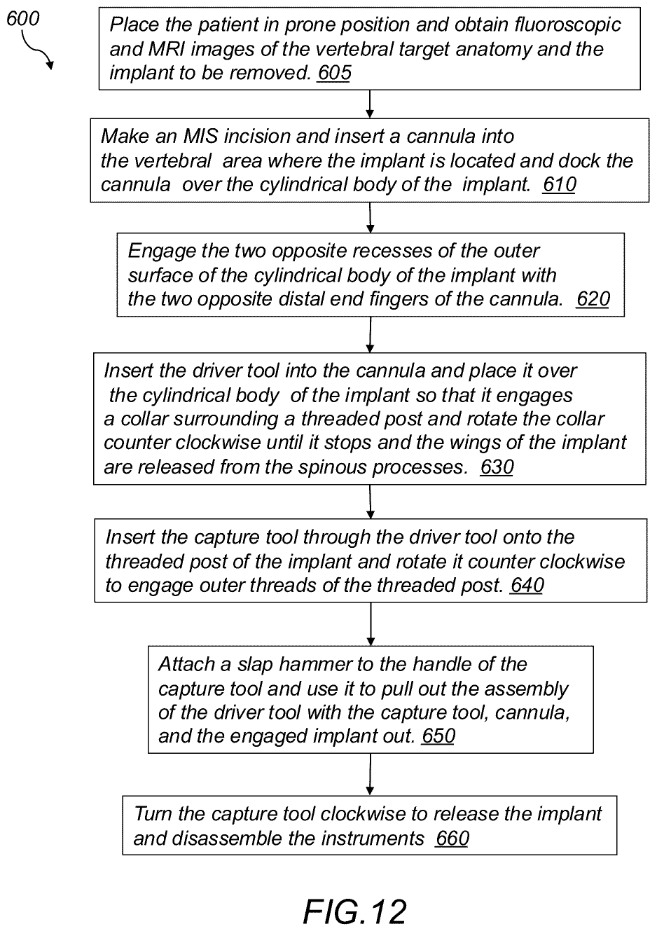

Referring to FIG. 12, the process 600 of removing the implant 140 from the surgical site includes the following steps. First, the patient is placed in the prone position and fluoroscopic and MRI images of the vertebral target anatomy and the implant are obtained (605). Next, an MIS incision is made and a cannula is inserted into the vertebral location where the implant 140 is located. The cannula is then docked over the cylindrical body of the implant (610). In some cannula tool embodiments, the distal end fingers of the cannula engage opposite outer recesses of the cylindrical body of the implant. In other cannula tool embodiments the distal end fingers of the cannula contour the cylindrical body of the implant (620). Next, the driver tool is inserted into the cannula and is placed over the cylindrical body of the implant so that it engages the implant wing release mechanism. In one embodiment, the implant wing release mechanism includes a threaded post with an affixed wedge and a collar around the threaded post. The driver tool includes distal protrusions that engage recesses formed on the collar around the threaded post of the implant. Next, the driver rotates the circular collar counter clockwise until it stops and the wings are released from the spinous processes (630). Next, the capture tool is inserted through the driver tool through-opening and the distal end of the capture tool is rotated counter clockwise to engage outer threads of the threaded post (640). Next, a slap hammer is used to pull the assembly of the cannula, the driver tool, the capture tool, and the engaged implant out of the surgical site (650). Finally, the capture tool is rotated clockwise to release the implant (660).

Several embodiments of the present invention have been described. Nevertheless, it will be understood that various modifications may be made without departing from the spirit and scope of the invention. Accordingly, other embodiments are within the scope of the following claims.

What is claimed is:

1. A device for spinal implant removal comprising:
a driver tool comprising an elongated cylindrical body with a through-opening and two distal end protrusions;
a capture tool comprising an elongated shaft configured to fit and extend through the cylindrical body through-opening of the driver tool and comprising a distal end opening with inner threads;
wherein the spinal implant comprises a cylindrical body and first and second sets of wings that are configured to engage first and second spinous processes of two vertebrae, respectively, and wherein the cylindrical body houses a threaded post that actuates the deployment of the first and second sets of wings;
wherein the two distal end protrusions of the driver tool are configured to engage a circular collar formed around the threaded post on a top surface of the cylindrical body and wherein the driver tool with the engaged circular collar are rotated counter clockwise to screw the threaded post out of the cylindrical body and thereby to release the first and second sets of wings; and
wherein the elongated shaft of the capture tool is configured to be rotated counter clockwise to capture outer threads of the threaded post with the inner threads of the distal end.

2. The device of claim 1, further comprising a cannula comprising an elongated body with a through-opening and two distal end protrusions, wherein the two distal end protrusions of the cannula are configured to engage two opposite recesses formed on an outer surface of the cylindrical body of the implant and wherein the driver tool is configured to be inserted into the cannula through-opening in order to reach the implant.

3. The device of claim 1, further comprising a cannula comprising an elongated body with a through-opening and two distal end protrusions, wherein the two distal end protrusions of the cannula are configured to wedge between an outer surface of the cylindrical body and the spinous processes.

4. The device of claim 3, wherein the cannula further comprises a distal end stop.

5. The device of claim 1, further comprising a slap hammer comprising an elongated shaft surrounded by a sliding weight, and wherein a distal end of the elongated shaft is configured to engage a proximal end of the capture tool.

6. The device of claim 5, wherein the distal end of the slap hammer comprises a channel that houses a spring plunger.

7. A method for spinal implant removal comprising:
providing a driver tool comprising an elongated cylindrical body with a through-opening and two distal end protrusions;
providing a capture tool comprising an elongated shaft configured to fit and extend through the cylindrical body through-opening of the driver tool and comprising a distal end opening with inner threads;
inserting the driver tool into a vertebral location where the spinal implant is located;
wherein the spinal implant comprises a cylindrical body and first and second sets of wings that are configured to engage first and second spinous processes of two vertebrae, respectively, and wherein the cylindrical body houses a threaded post that actuates the deployment of the first and second sets of wings;
engaging the two distal end protrusions of the driver tool to a circular collar formed around the threaded post on a top surface of the cylindrical body;
rotating the driver tool with the engaged circular collar counter clockwise to screw the threaded post out of the cylindrical body and thereby to release the first and second sets of wings;
inserting the elongated shaft of the capture tool into the vertebral location where the spinal implant is located through the through-opening of the cylindrical body of the driver tool; and
rotating the elongated shaft of the capture tool counter clockwise to capture outer threads of the threaded post with the inner threads of the distal end.

8. The method of claim 7, further comprising pulling a proximal end of the elongated shaft of the capture tool to remove the implant, the capture tool and the driver tool.

9. The method of claim 7, further comprising providing a cannula comprising an elongated body with a through opening and two distal end protrusions, wherein the two distal end protrusions of the cannula are configured to engage two opposite recesses formed on an outer surface of the cylindrical body of the implant, and wherein the driver tool is configured to be inserted into the cannula through-opening in order to reach the implant.

10. The method of claim 7, further comprising providing a cannula comprising an elongated body with a through opening and two distal end protrusions, wherein the two distal end protrusions of the cannula are configured to wedge between an outer surface of the cylindrical body and the spinous processes, and wherein the driver tool is configured to be inserted into the cannula through-opening in order to reach the implant.

11. The method of claim 10, wherein the cannula further comprises a distal end stop.

12. The method of claim 7, further comprising providing a slap hammer comprising an elongated shaft surrounded by a sliding weight, and engaging a distal end of the elongated shaft to a proximal end of the capture tool.

13. The method of claim 12, wherein the distal end of the slap hammer comprises a channel that houses a spring plunger.
